(19) United States
(12) Patent Application Publication
(10) Pub. No.: US 2005/0025812 A1
(43) Pub. Date: Feb. 3, 2005

(54) Salad dressing with weight loss supplement

(76) Inventor: Carl A. Forest, Boulder, CO (US)

Correspondence Address: PATTON BOGGS, 1660 LINCOLN ST, SUITE 2050, DENVER, CO, 80264, US

(21) Appl. No.: 10/616,674

(22) Filed: Jul. 10, 2003

(51) Int. Cl. A61K 35/78; A61K 47/00

(52) U.S. Cl. 424/439; 424/776

(57) ABSTRACT

A weight loss salad dressing including a salad dressing base and a weight loss supplement. The weight loss supplement may be a fat or carbohydrate blocker, an appetite suppressant, a metabolizer or thermogenic agent, a weight loss stimulant, a nutrient partitioning modulator, or any other substance which is ingested not directly for its food value, but to interact with the body chemistry to prevent other substances with food value from being eaten, to cause other substances with food value to be eliminated without being absorbed, to cause the body to burn more fat or carbohydrate, or to cause the body to metabolize food differently. For the best taste, if the weight loss supplement is acidic, the acidity of the salad dressing base is adjusted to account for the acidity of the supplement.

### BACKGROUND OF THE INVENTION

1. Field of the Invention

The invention relates to foods and particularly salad dressings with added nutritional ingredients.

2. Statement of the Problem

Obesity is a serious health problem in the United States and many other countries. About 90 million citizens are now considered obese. Obesity is known to be associated with many serious diseases and metabolic disorders including small, dense LDL syndrome, glucolipoxia, premature aging, memory loss, endothelial dysfunction, vascular disease, hypertension, postprandial hyperlipidemia, insulin resistance, hyperinsulinemia, Syndrome X, hypertriglyceridemia and/or low HDL syndrome, high RQ (respiratory quotient) syndrome, chronic fatigue syndrome, as well as certain types of cancer. In addition, it has psychological implications.

Because of the recognized problem of obesity, many food supplements and medications have been developed to address the problem. Nearly every avenue of weight loss has been explored with these supplements and medications. Appetite suppressants attack the problem by preventing overeating. Stimulants address the problem by causing the user to burn more energy. More recently fat and/or carbohydrate blockers have become available which prevent the digestion and/or absorption of fat and/or carbohydrates in the intestines. A more sophisticated approach utilizes thermogenic agents to alter the metabolism to cause the body to burn more energy. A related relatively sophisticated approach starts from the premise that the metabolic functions of our bodies are not well adapted to current dietary and lifestyle choices. That is, the human body that was developed over a period when humans had to expend large amounts of energy to gather or hunt for food, and lived in unheated buildings requiring further expenditures of energy to stay warm, may be maladapted for a sealed, controlled environment and a lifestyle that requires sitting before a computer for long periods and provides inexpensive fast foods. These approaches study the metabolic system and provide supplements that adjust the human metabolic system to better fit current diets and lifestyles. See, for example, U.S. Pat. No. 6,579,869 B2, which issued Jun. 17, 2003 to Larry McCleary, which is directed to a method and a nutritional supplement composition for modulating nutrient partitioning in the body so as to increase oxidation of fat and promote increased storage of glycogen. Another weight loss composition designed to reduce synthesis of fats is disclosed in U.S. Pat. No. 5,626,849 issued May 6, 1997 to Hastings et al. U.S. Pat. No. 6,020,378 issued Feb. 1, 2000 to Cook et al. discloses a method for selectively altering body fat levels in animals involving administering to the animal a combination of conjugated linoleic acid isomers in a ratio selected to retain a desirable benefit attributable to one isomer while counteracting an undesirable effect of the same isomer.

The above are just a few of the weight loss nutritional supplements that are known in the art. Yet, the obesity problem continues to grow. In 1990, one in four Americans was considered obese. Now, the ratio is one in three. For any of the above supplements to succeed, the overweight person must keep a supply of the supplements available and remember to take them regularly. Though in theory this may seem easy to do, in practice it is not. Most homes have cupboards where food supplements sit unused because they are forgotten in the everyday pace of life. Meals are often had away from home or on business, so if an overweight person is to use a supplement regularly, he or she must carry it with them at all times, which is inconvenient. If they do carry it with them, it may be sitting in their car when they find themselves in a restaurant. Even if the supplement is on the person, many would not use them in a social or business meal situation.

As a result, obesity tends to be a chronic problem for many people. The problems are ongoing and require ongoing treatment. Some people, for example, struggle against obesity and related overweight problems for much of their life. Thus, it would be highly desirable to have a weight loss product that is transparently easy to use and was available in the course of everyday life.

### SUMMARY OF THE INVENTION

The invention arose out of personal experience of the difficulty of having food supplements available in a fast-paced life style, the realization that many weight loss supplements taste somewhat acidic and thus blend well with salad dressings, and the observation that salads are everywhere. Every restaurant today has a variety of salads on the menu, so much so that salads are now viewed not as a pre-course but as a main course. Fast food restaurants such as McDonalds™ and Wendys™ offer excellent salads as part of their menus. Thus, if a salad dressing could be provided that included weight loss food supplements, people would find it much easier to regularly ingest these supplements.

The primary object of this invention is to provide a salad dressing that includes a weight loss food supplement. The term “weight loss food supplement” does not include substitutes for fat, oil, or sugar, but is limited to substances that directly affect body chemistry to bring about weight loss. These include any and all substances generally known as weight loss supplements, including, but not limited to, fat blockers, carbohydrate blockers, appetite suppressants, metabolic agents, thermogenic agents, weight loss stimulants, nutrient partitioning modulators, and any other substance which is ingested not directly for its food value, but to interact with the body chemistry to prevent other substances with food value from being eaten, to cause other substances with food value to be eliminated without being absorbed, to cause the body to burn more fat or carbohydrate, or to cause the body to metabolize food differently.

In the preferred embodiment, the invention provides a salad dressing composition and method for modulating nutrient partitioning in the body so as to increase oxidation of fat.

One preferred embodiment of the invention comprises garcinia camobogia or a derivative thereof, aspartic acid, L-carnitine, biotin, and chromium mixed with a salad dressing base.

The invention provides a weight loss salad dressing comprising: a salad dressing base; and a weight loss supplement, which weight loss supplement is present in an amount in excess of the amount of said supplement present naturally in foods. The weight loss supplement can comprise a fat blocker, a carbohydrate blocker, a fat and carbohydrate blocker, an appetite suppressant, a metabolizer, a thermogenic agent, a weight loss stimulant, or nutrient partitioning modulator. Preferably, the weight loss supplement comprises one or more of the following ingredients: mahuang or a derivative thereof, guarana seed or a derivative thereof, L-aspartic acid, L-carnitine, garcinia cambogia or a derivative thereof, and hydroxycitric acid. Preferably, said salad dressing base comprises a salad dressing base selected from the group consisting of: an oil and vinegar salad dressing base, a Caesar salad dressing base, a French salad dressing base, a ranch salad dressing base, a bleu cheese salad dressing base, a Russian salad dressing base, and a Thousand Island salad dressing base. Preferably, the weight loss salad dressing further includes one or more ingredients selected from a preservative, a color enhancer, a thickening agent, a vitamin, a mineral, and an inactive ingredient. Preferably, the acidity of said salad dressing base is adjusted for any acidity of said weight loss supplement. Preferably, said weight loss supplement comprises: an effective amount of hydroxycitric acid; an effective amount of carnitine; an effective amount of biotin; an effective amount of one or more gluconeogenic substrates selected from the group consisting of: aspartate, lactate, glycerol; and a gluconeogenic amino acid or an alphaketo analogue thereof. Preferably, said weight loss supplement comprises: a weight ratio of the hydroxycitric acid to the carnitine of from about 1:10 to about 100:1; a weight ratio of the hydroxycitric acid to the gluconeogenic substrate of from about 5:1 to about 1:60; and a weight ratio of the hydroxycitric acid to the biotin of from about 50:1 to about 2500:1. Preferably, the gluconeogenic amino acid is selected from the group consisting of: alanine, arginine, asparagine, cystine, glutamine, glycine, histidine, hydroxyproline, methionine, proline, serine, threonine, and valine. Preferably, said weight loss supplement further comprises an effective amount of at least one nutritional supplement. Preferably, the nutritional supplement is selected from the group consisting of: chromium, conjugated linoleic acid, coenzyme Q10, eicosapentaenoic acid, pyridoxine, alpha-lipoic acid, magnesium, and gymnema sylvestre. Preferably, said weight loss supplement comprises: from about 0.2 grams to about 8 grams of hydroxycitric acid; from about 10 milligrams to about 10 grams of carnitine; from about 1 gram to about 75 grams of the gluconeogenic substrate; from about 1 milligram to about 25 milligrams of biotin; from about 100 micrograms to about 2 milligrams of chromium; from about 5 milligrams to about 500 milligrams of coenzyme Q10; from about 50 milligrams to about 20 grams of conjugated linoleic acid; from about 10 milligrams to about 10 grams of eicosapentaenoic acid; from about 25 milligrams to about 400 milligrams of pyridoxine; from about 25 milligrams to about 2000 milligrams of alpha lipoic acid; from about 200 milligrams to about 1600 milligrams of magnesium; and from about 20 milligrams to about 2000 milligrams of gymnemic acid. In another embodiment, said weight loss supplement comprises: from about 0.5 grams to about 5 grams of hydroxycitric acid; from about 50 milligrams to about 5 grams of carnitine; from about 1 gram to about 30 grams of the gluconeogenic substrate; from about 2 milligrams to about 10 milligrams of biotin; from about 400 micrograms to about 2400 micrograms of chromium; from about 20 milligrams to about 300 milligrams of coenzyme Q10; from about 1 gram to about 10 grams of conjugated linoleic acid; from about 50 milligrams to about 5000 milligrams of eicosapentaenoic acid, from about 50 milligrams to about 300 milligrams of pyridoxine; from about 50 milligrams to about 1200 milligrams of alpha lipoic acid; from about 400 milligrams to about 1200 milligrams of magnesium; and from about 75 milligrams to about 500 milligrams of gymnemic acid.

The invention also provides a method of effecting weight loss in a human being, said method comprising ingesting, for a therapeutically effective period of time, an effective amount of a weight loss salad dressing comprising a salad dressing base and a weight loss supplement, which weight loss supplement is present in an amount in excess of the amount of said supplement present naturally in foods. Preferably, the method further includes said human following a dietary regimen involving a glycemic index of less than 60 and daily calorie consumption comprising less than 50% of calories from carbohydrate intake and at least 20% of calories from protein intake. Preferably, the method further includes said human following an exercise program involving aerobic and resistance training. Preferably, the method further involves said human donating blood so as to produce a fall in serum ferritin levels and iron stores. Preferably, the method further involves said human following a stress reduction program so as to diminish glycocorticoid activity.

Numerous other features, objects and advantages of the invention will become apparent from the following description when read in conjunction with the accompanying drawing.

### BRIEF DESCRIPTION OF THE DRAWING

FIG. 1 is a block diagram illustrating the weight loss salad dressing according to the invention and the method of making it.

### DETAILED DESCRIPTION OF THE PREFERRED EMBODIMENT

1. Overview

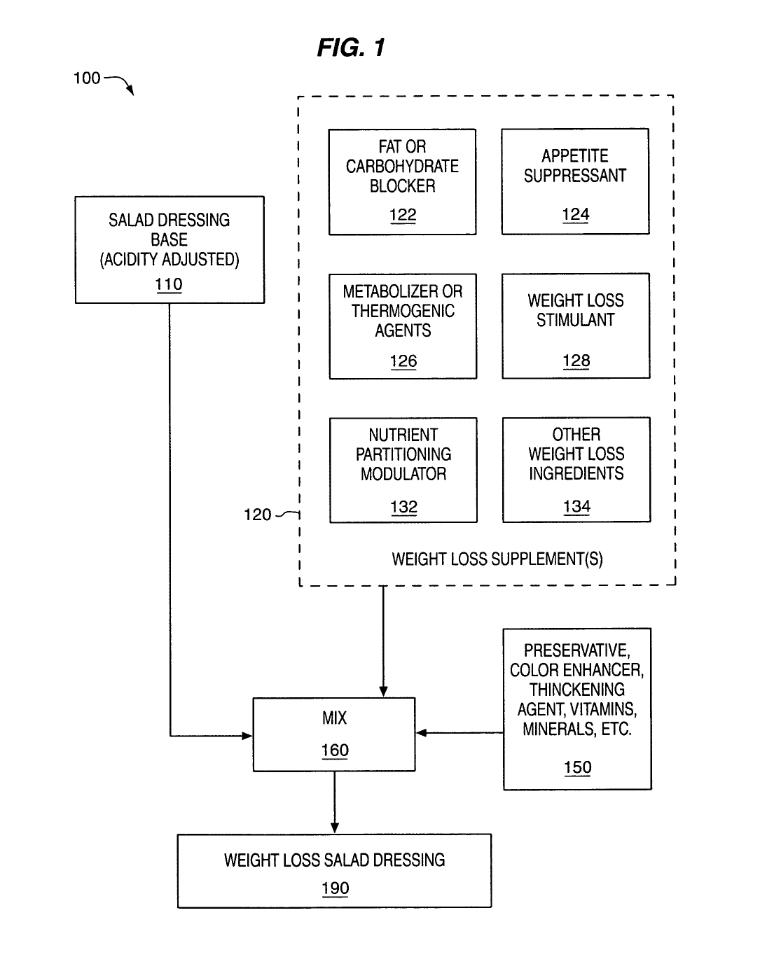

FIG. 1 illustrates the weight loss salad dressing 190 according to the invention and the method of making it. The weight loss salad dressing includes a salad dressing base 110 and a weight loss supplement 120. Optionally, it may also include other ingredients 150, such as preservatives, color enhancers, thickening agents, vitamins, minerals, or other ingredients which may be added to foods. Salad dressing base 110, weight loss supplement 120, and any other desirable ingredient 150 are mixed at 160 to create weight loss salad dressing 190.

The salad dressing bases disclosed herein are all formulations that are considered to be “normal” salad dressings, as compared to low-fat or light (or lite) salad dressings. However, the “term salad dressing base” as used herein is intended to include any and all low-fat or light salad dressing bases also. A low-fat or light salad dressing may be derived from any of the salad dressing bases given below by substituting water and a thickening agent, such as xanthan gum, for all or a portion of the oil given in the formulation, or by other substitutions and formulations known in the salad dressing art.

The terms “weight loss supplement” or “weight loss ingredient” as used herein include only substances or formulations which are recognized as weight loss supplements in the art of weight loss food supplements. They do not include diet ingredients such as low-fat or light formulations or sugar substitutes. That is, low-fat or light formulations of salad dressing are included in the salad dressing bases. Generally, weight loss supplements or weight loss ingredients are substances or formulations that are intended to affect body chemistry as opposed to diet ingredients which are intended to affect the amount of calories a food contains. Weight loss supplements 120 are divided into six groups: fat or carbohydrate blockers; appetite suppressants; metabolizers or thermogenic agents; weight loss stimulants; nutrient partitioning modulators; and other weight loss ingredients. The groups selected are those commonly used in the art of weight loss supplements and are provided to make it easier to understand the invention. There is overlap between the groups. That is, those skilled in the art of weight loss supplements tend to use different terminology, depending on their point of view. For example, there is considerable overlap between the metabolizer or thermogenic agent group and the nutrient partitioning modulator group. Therefore, we have included the common terminologies used in the art. Other terminologies certainly are used, and more may develop in the future, so we have also included an “other” group for completeness. In this disclosure, all terms that relate to the salad dressing component have the meaning commonly used in the food art, and all terms that relate to the weight loss component have the meaning they commonly have in the weight loss art. For example, “weight loss stimulant” excludes things such as eye stimulants and includes only stimulants that are specifically adapted to directly stimulate organs, such as the thyroid, brain, or nervous system in such a manner as to lead to weight loss.

In the following, we will disclose a number of different salad dressing bases and a number of different weight loss supplements that have been found to be useful to make weight loss salad dressing product 190. However, it should be understood that these are exemplary, that is, only illustrative, and not intended to be exhaustive.

In the following, the amounts of ingredients are given to make a small bottle of about twelve to fifteen ounces of weight loss salad dressing. It is intended that the amounts of the ingredients given in the various salad dressing bases should be mixed with the amounts of the ingredients given in the various weight loss supplements. The amounts are designed so that a serving of salad dressing is two ounces. It is assumed that salads are eaten twice a day so that two salads, or four ounces of salad dressing, provide effective daily amounts of weight loss ingredients. The various amounts given for salad dressing bases may be varied as known in the art of salad dressings, and the various amounts given for weight loss ingredients may be varied as known in the art of weight loss supplements. In the following, “tsp” means teaspoon, “tbsp” means tablespoon, and “mg” is milligrams.

2. Salad Dressing Bases

When vegetable oil is mentioned, any vegetable oil may be used, preferably soybean oil or canola oil.

TABLE A
Oil And Vinegar Salad Dressing Base

½ cup water
½ cup vegetable oil
¼ cup vinegar (red wine vinegar may be used)
1 tsp sugar (optional)
½ tsp salt
½ tsp pepper

Small amounts of spices such as onion powder and/or garlic powder may be added.

TABLE B
Caesar Salad Dressing Base

1 egg, raw
3 tbsp lemon juice
garlic (about ½ teaspoon garlic powder or a large clove)
1 cup olive oil (other vegetable oil may be used)
¼ to ½ cup grated Parmesan or Romano cheese
1 tsp high fructose corn syrup
4 tbls anchovy paste
¼ tsp salt and pepper to taste

TABLE C
French Salad Dressing Base

½ cup vegetable oil
⅓ cup high fructose corn syrup
¼ cup water
3 tbls vinegar
½ tsp salt
½ tsp whey
⅓ tsp modified food starch
pinch paprika
Enough yellow food color #5 and/or #6 to give desired color

TABLE D
Ranch Salad Dressing Base

½ cup water
½ cup vegetable oil
3 tbls vinegar
3 tbls sugar
1 egg yolk
2 tbls buttermilk
½ tsp salt
½ tsp whey
½ tsp modified food starch
¼ teaspoon malted dextrin

TABLE E
Bleu Cheese Salad Dressing Base

½ cup vegetable oil
¼ cup vinegar
¼ cup water
3 tbls blue cheese
2 tbls high fructose corn syrup
1 egg yolk
1 tsp lactic acid

TABLE F
Russian Salad Dressing Base

½ cup high fructose corn syrup
½ cup vegetable oil
½ cup water
2 tbls tomato paste
2 tbls vinegar
1 tbls sugar
½ tsp salt
½ tsp malted dextrin
½ tsp lemon juice
¼ tsp yeast extract
¼ tsp orange juice
pinch of paprika

TABLE G
Thousand Island Salad Dressing Base

½ cup water
¼ cup tomato paste
¼ cup high fructose corn syrup
¼ cup vegetable oil
2 tbls vinegar
1 tbls chopped pickles
¼ tsp salt
⅛ tsp turmeric

The above oil and vinegar salad dressing base, Caesar salad dressing base, French salad dressing base, Ranch salad dressing base, Bleu Cheese salad dressing base, Russian salad dressing base, and Thousand Island salad dressing base are merely exemplary salad dressing bases. As is known in the art, there are many other variations of salad dressing bases, and particularly many other variations of oil and vinegar salad dressing bases, Caesar salad dressing bases, French salad dressing bases, Ranch salad dressing bases, Bleu Cheese salad dressing bases, Russian salad dressing bases, and Thousand Island salad dressing bases, all of which may be used in the invention.

3. Weight Loss Supplements

A. Fat and/or Carbohydrate Blockers

TABLE I

Orlistat - 1 gram to 2 grams preferred; 1.4 gram to 1.5 gram
most preferred. This is a prescription product available, for
example, under the name Xenical ™.

TABLE II
Natural Carbohydrate and fat blocker

```
0.72 grams Vitamin C
  6 grams Phaseolus vulgaris (northern white kidney bean) extract
  6 grams marine fiber concentrate
192 mg Lagerstoemia speciosa leaf extract
  3 grams Gymnema sylvestre leaf extract
  0.6 grams bitter melon fruit extract
  0.1 grams Vanadium
```

The above are the most preferred amounts. The amounts preferably can range from one-fifth to five times the most preferred amounts. Any commercially available fat and/or carbohydrate blocker, such as Chitosol™, Chitosan™, and C-block™, may be used in amounts indicated by their manufacturers.

B. Appetite Suppressant

TABLE III

Phentermine HCL - 100 mg to 300 mg preferred. 200 mg
most preferred. This is a prescription medicine that is available
under brand names such as Adipex-P ™. Any other prescription
appetite suppressant such as phendimetrizine, sibutramine,
benzphematamine, and Diethylpropion which are available under
brand names Bontril ™, Meridia ™, Didrex ™, and Tenuate ™,
respectively, and many other such appetite suppressants may be
used in appropriate amounts.

TABLE IV
Natural Appetite Suppressant

Guarana extract (22%) - extracted from the seed of the tropical
fruit Paullina cupana var. sorbilis. Preferred amount is 1 gram to 20
grams, and most preferably 5 grams.

C. Metabolizer or Thermogenic Agent

TABLE V
Preferred Natural Thermogenic Formula

| | |
|---|---|
| 3 | grams Garcinia Cambogia fruit extract |
| 24 | grams Aspartic Acid |
| 60 | mg L-Carnitine (preferably, L-carnitine L-tartrate) |
| 3.6 | mg Biotin |
| 2.4 | mg Chromium |

TABLE VI
Alternative Natural Thermogenic Formula

| | |
|---|---|
| 0.9 | grams mahuang extract containing 72 mg ephedrine alkaloids |
| 3 | grams Guarana seed extract containing 0.6 grams of caffeine |
| | alkaloids |
| 0.6 | grams white willow bark extract |
| 1.8 | grams citrus aurantium extract |
| 3 | grams Garcinia cambogia fruit extract |
| 150 | mg Soybean lecithin |
| 150 | mg Uva Ursi leaf |
| 100 | mg L-carnitine (preferably, L-carnitine L-tartrate) |

Other typical ingredients used in fat metabolizers and thermogenic agents include lipo-chromizyme, green tea extract, citrus aurantium, hydroxycitric acid, chromium picolinate, and 3-acetyl-7oxo-dehydroepiandrosterone. The invention contemplates that any commercially available metabolizer or thermogenic agent may be used, such as Xenadrine™, Metabolift™, Metabolic Trim 2000™, Fat Ignite™, and Zantrex-3™, in amounts given by their manufacturers.

D. Weight Loss Stimulants—See Tables IIII and IV, Appetite Suppressants Above. Most Appetite Suppressants are Weight Loss Stimulants.

E. Nutrient Partitioning Modulator

This is the most preferred weight loss supplement formulation and therefore it will be discussed in some detail. The composition of the nutrient partitioning modulator is preferably composed of effective amounts of: HCA (hydroxy citrate); carnitine (sometimes spelled “carnatine”); biotin; and a gluconeogenic substrate preferably selected from one or more of the following: aspartate, lactate, glycerol, and any gluconeogenic amino acid or its alpha-keto analogue. The gluconeogenic amino acid is preferably alanine, arginine, asparagine (aspartic acid), cystine, glutamine, glycine, histidine, hydroxyproline, methionine, proline, serine, threonine and valine. The preferred form of L-carnitine is L-carnitine L-tartrate, which is a form of L-carnitine made by Lonza Group Ltd., Switzerland. This ingredient has a pleasant citrus taste and is therefore particularly well suited as a supplement to salad dressings.

Preferably, the composition of the nutrient partitioning modulator further contains one or more of the following: chromium, CLA (conjugated linoleic acid), coenzyme Q10, EPA (eicosapentanoic acid) (either alone or as part of fish oil), pyridoxine, alpha lipoic acid, magnesium, and Gymnema sylvestre.

HCA and carnitine promote hepatic fatty acid oxidation by activating carnitine palmitoyl transferase (CPT), the rate-limiting enzyme in the fatty acid oxidation process.

EPA (either alone or as part of fish oil) inhibits the enzymes citrate lyase (CL) and acetyl CoA carboxylase (ACC), thus inhibiting the production of malonyl CoA, an allosteric inhibitor of CPT. EPA/fish oil thus acts to disinhibit fatty acid oxidation.

As one of its metabolic effects, CLA promotes fat-to-lean partitioning changes via the activation of CPT.

Thus, HCA, carnitine, EPA, and CLA promote hepatic fatty acid oxidation.

Hepatic fat oxidation produces reducing equivalents, ATP, and acetyl CoA which drive hepatic gluconeogenesis. The elevated acetyl CoA levels activate the liver enzyme pyruvate carboxylase (PC) and high ATP levels inhibit pyruvate dehydrogenase (PDH). These effects synergistically accelerate hepatic gluconeogenesis while simultaneously inhibiting fat synthesis. The reducing equivalents generated by disinhibited fatty acid oxidation are consumed in the reductive synthetic processes of gluconeogenesis.

Hepatic gluconeogenesis acts via the indirect route to expand liver glycogen stores and provides a slow continuous time-release source of glucose from the liver. Both of these have the physiologic effect of profoundly suppressing appetite and increasing energy. These effects are enhanced by the consumption of appropriate amounts of low glycemic index carbohydrates whose glucose content is absorbed slowly over a prolonged time interval, thus acting as a sustained release glucose source emanating from the gut.

It has also been shown that hepatic triglyceride (TG) synthesis and very low density lipoprotein (VLDL) secretion are diminished coincidentally with increasing hepatic fatty acid (FA) oxidation.

Coenzyme Q10 facilitates respiratory chain function and hence augments the process of reverse electron transport. This process plays a key role in the thermogenic effect produced by accelerated fatty acid oxidation.

Biotin induces the up regulation of the enzyme glucokinase (GK) in the pancreas and enhances the process of glucose-induced insulin secretion. In addition, biotin counteracts the effect HCA has on the pancreas. HCA tends to inhibit glucose-induced insulin secretion, while biotin facilitates it by activating GK. Together, HCA and biotin maximize pancreatic fuel homeostasis.

Pyridoxine supplementation augments physiological levels of pyridoxal phosphate. This compound interacts with glucocorticoid receptors to down-regulate their activity and hence to diminish glucocorticoid effects throughout the body. This action tends to decrease visceral fat accumulation as well as promote insulin sensitivity. Improved insulin sensitivity contributes to insulin-induced appetite suppression. In addition, a direct consequence of improved insulin sensitivity is a decrease in the activity of insulin like growth factor (IGF), a potent cancer inductive agent.

Chromium improves insulin sensitivity in the central nervous system and skeletal muscle. The former contributes to insulin-induced appetite suppression at the level of the hypothalamus while the latter improves insulin-induced glucose disposal into skeletal muscle. In the present invention, chromium can be added in the form of a non-toxic salt, such as, e.g., chromium diglycinate, chromium arginate, chromium polynicotinate, and the like.

CLA, alpha lipoic acid, Gymnema sylvestre, coenzyme Q10, and magnesium each have insulin sensitizing effects. In the nutrient modulator, magnesium can be added in the form of a non-toxic salt.

With respect to the amounts of the individual components of the composition of the nutrient partitioning modulator, the term “effective amount” means that amount of the component which, when used in combination with the other components in the composition, will provide the composition with the capability of modulating nutrient partitioning so as to increase oxidation of fat and promote increased storage of glycogen.

Preferably, the composition contains an HCA:carnitine weight ratio of from about 1:10 to about 100:1; an HCA:biotin weight ratio of from about 50:1 to about 2500:1; and an HCA:gluconeogenic substrate weight ratio of from about 5:1 to about 1:60.

Set forth in Table VII below is a preferred embodiment of the composition (excluding inactive ingredients) of the nutrient partitioning modulator. Table VIII sets forth a more preferred embodiment of the composition. The amounts recited in Tables VII and VIII represent the preferred amounts of the ingredients listed which are to be added to the salad dressing bases described above.

TABLE VII
Preferred Nutrient Partitioning Modulator Ingredients and Amounts

| | |
|---|---|
| 6 grams-450 grams | Gluconeogenic Substrate |
| 1.2 grams-33 grams | HCA |
| 30 mg-30 grams | Carnitine |
| 2 mg-150 mg | Biotin |
| 0.6 mg-12 mg | Chromium |
| 0.3 grams-120 grams | CLA |
| 60 mg-60 grams | EPA (alone or as a component of fish body oil) |
| 30 mg-3 grams | Coenzyme Q10 |
| 0.15 grams-12 grams | Alpha Lipoic Acid |
| 1.2 grams-9.6 grams | Magnesium |
| 0.10 grams-2.4 grams | Pyridoxine |
| 0.30 grams-12 grams | (from Gymnema sylvestre) |

TABLE VIII
More Preferred Nutrient Partitioning Modulator Amounts

| | |
|---|---|
| 6 grams-180 grams | Gluconeogenic Substrate |
| 1.5 grams-30 grams | HCA |
| 60 mg-30 grams | Carnitine |
| 3 mg-60 mg | Biotin |
| 1.2 mg-6 mg | Chromium |
| 0.5 grams-60 grams | CLA |
| 0.3 grams-30 grams | EPA (alone or as a component of fish body oil) |
| 60 mg-2.4 grams | Coenzyme Q10 |
| 0.3 grams-4.8 grams | Alpha Lipoic Acid |
| 1.2 grams-7.2 grams | Magnesium |
| 0.15 grams-1.8 grams | Pyridoxine |
| 0.45 grams-3 grams | Gymnemic Acid (from Gymnema sylvestre) |

TABLE IX
Alternative Preferred Nutrient Partitioning Modulator

| | |
|---|---|
| 1-50 grams | L-Aspartic Acid - most preferably 20 grams |
| 1-6 grams | Garcinia cambogia - most preferably, 3 grams |
| 0.1 grams-30 grams | L-Carnitine (preferably, L-carnitine L-tartrate) - |
| | most preferably, 1 gram. |
| 30 mg-600 mg | Ribovflavin - most preferably, 150 mg |
| 0.1 mg-25 mg | Biotin - most preferably, 3.6 mg |
| 0.1 mg-20 mg | Chromium polynicotinate - most preferably, 1.5 mg |

As used herein with respect to the amount of the composition used in the method of this invention, the term “effective amount” means an amount sufficient to modulate nutrient partitioning in the body so as to increase oxidation of fat and increase storage of glycogen. Preferably, the active-ingredient composition (not including inactive ingredients) of the nutrient partitioning modulator is a per serving dosage of at least about 0.5 gram, more preferably from about 0.5 gram to about 100 grams, and most preferably from about 2 grams to about 20 grams.

The term “therapeutically effective period of time” with respect to the ingestion of the composition in the method of this invention means that period of time sufficient to modulate nutrient partitioning in the human. Preferably, the weight loss salad dressing is eaten on a daily basis for a period of at least three weeks, and more preferably at least six weeks. Most preferably, it is eaten twice a day.

The method of the invention preferably involves the ingestion preferably of the above-described weight loss supplement in conjunction with a specific dietary plan in a synergistic format. The dietary plan preferably involves multiple small meals, each reflecting the overall macronutrient composition of the diet. Carbohydrate content is low, as is the glycemic index. Protein content is high. In the dietary regimen followed in the present invention, the glycemic index is preferably less than 60, and more preferably less than 45; the carbohydrate content will constitute less than 50%, more preferably 0% to 50%, and most preferably about 7% to 40% of the calories consumed on a daily basis; and the protein intake constitutes preferably at least 20%, more preferably about 20% to 40%, and most preferably about 25% to 35% of total daily caloric intake. The number of meals is preferably 2, more preferably 4 to 6, per day. Adequate fluid intake is recommended to insure excellent hydration.

As stated above, the weight loss salad dressing of this invention is preferably ingested on a daily basis. However, its use on a non-daily basis, e.g., every other day, is still effective. The frequency of use will depend on how fast the individual wishes to lose weight. The more frequently the weight loss salad dressing is used, the faster the weight loss. Thus, daily use of the composition will result in faster weight loss than non-daily use.

Low glycemic index, low carbohydrate, high protein diets have the physiologic effect of inducing low serum insulin (I) levels and low serum insulin/glucagon (I/G) ratios. Both of these parameters act to disinhibit hepatic fatty acid oxidation by decreasing malonyl CoA levels and desensitizing CPT to inhibition by malonyl CoA. On the other hand, if a high carbohydrate diet (greater than 50% carbohydrates) is consumed, average daily insulin levels and the I/G ratio are increased, thereby adversely impacting CPT activity and acting to inhibit fat oxidation.

The method of this invention preferably further includes an exercise program. Preferably, the exercise program will be followed at least 2 days a week and more preferably 3 days to 5 days per week. The exercise program should preferably include components of aerobic and resistance training as tolerated by the individual in need thereof.

The exercise program augments fatty acid oxidation during the period of active exercise, as well as inducing a fall in RQ post exercise. Insulin sensitivity is increased after each bout of exercise, as well as with exercise training. Exercise programs produce changes parallel to those described herein which augment insulin sensitivity, facilitate energy expenditure, and reduce RQ for an extended period of time post exercise.

The invention may further include a stress reduction program designed to diminish glucocorticoid activity. By downgrading glucocorticoid activity, the stress reduction program acts to improve insulin sensitivity and decrease visceral obesity. Utilizing similar mechanisms, pyridoxine augments both these actions. The stress reduction program may involve any activity that lowers glucocorticoid levels. Non-limiting examples of such activities include relaxation, getting a massage, acupuncture, psychotherapy, meditation, taking a sedative, and the like.

The method of the present invention may also include a blood donation program. Blood may be given about every 56 days. This produces a fall in both serum ferritin levels and iron stores within the body, which together decrease oxidative stress and improve insulin sensitivity. The amount of blood donated will depend on the individual's serum ferritin levels. Generally, the frequency and amount of blood donated should be such as to provide a serum ferritin level of from about 25 nanograms/milliliter to about 50 nanograms/milliliter of serum.

The method of this invention, particularly the use of the preferred embodiments of the weight loss salad dressing according to the invention, results in marked increases in fat oxidation and glycogen storage while simultaneously minimizing fat synthesis and storage.

Without being bound by any particular overall mechanistic explanation of the invention, the effects of the present invention upon hormone levels and ratios (i.e., low I level, low I/G ratio) facilitate the release of fatty acids for presentation at the hepatocyte mitochondrial membrane. At this locus resides the enzyme CPT. As stated previously herein, CPT is the rate-limiting enzyme in the oxidation of activated long chain fatty acids. Carnitine represents the essential cofactor for CPT and is generally in the sub-saturating range in the liver. Exogenous supplementation of carnitine augments CPT activity. Low I levels and a low I/G ratio tend to cause carnitine uptake and concentration in hepatocytes, which in turn acts to increase CPT activity in the liver.

Malonyl CoA, a potent inhibitor of CPT, is a cytosolic metabolite derived from citrate. The cytosolic enzymes CL and ACC are involved in metabolizing cytosolic citrate to acetyl CoA and then on to malonyl CoA, respectively. HCA is a potent competitive inhibitor of CL, thus acting to decrease malonyl CoA levels. EPA/fish oil decreases the activity of both CL and ACC and makes CPT less sensitive to the inhibitory effects of malonyl CoA. The effect of the low I level and low I/G ratio resulting from the dietary plan additionally diminishes the activity of CL and ACC and has a similar desensitizing effect on CPT to the inhibitory action of malonyl CoA. CLA also serves to activate CPT. The action derived from the combined effect of these mechanisms profoundly disinhibits fatty acid oxidation.

Hepatic oxidation of fatty acids to acetyl CoA proceeds independently of the rate of generation of ADP by the liver. This induces the production of mitochondrial acetyl CoA and ATP at high rates. Reducing equivalents in the form of NADH and FADH₂ are also abundantly generated. These effects combine to upregulate reverse electron transport—a highly thermogenic process. In addition, the enzyme PC is activated by the production of high levels of acetyl CoA. At the same time, the enzyme PDH is inhibited by the high ATP levels. Together, these actions channel substrate into the gluconeogenic pathways and away from fat synthetic pathways. The reducing equivalents generated by activation of the fatty acid oxidation process also drive gluconeogenesis. The gluconeogenic process is also thermogenic and is coupled with replenishment of hepatic glycogen stores via the indirect pathway as well as enhanced hepatic glucose output—a process that provides a continuous slow time-release source of serum glucose. The gluconeogenesis substrate used in the invention acts as substrate for the gluconeogenic pathway, further acting to facilitate gluconeogenesis. Because the gluconeogenesis substrate is provided exogenously, it tends to spare muscle protein breakdown, thus acting in an anticatabolic fashion and promoting expansion of lean tissue mass. The net effect of this combination of metabolic actions is to profoundly suppress appetite while increasing energy levels, and at the same time oxidizing fat at high rates in a thermogenic fashion. These combine to decrease energy intake while enhancing energy expenditure. This causes a significant depression of RQ as a reflection of the profound alteration in fuel homeostasis and nutrient partitioning which are induced. This lowers the risk of recurrent weight gain following prior weight loss.

F. Other Weight Loss Ingredients

Most, if not all, weight loss supplements fall into one of the above categories. However, many names are used for the various chemical substances that have weight loss properties. Because a supplement is called by some other name, this does not take it outside the invention if it functions as a fat blocker, carbohydrate blocker, appetite suppressant, metabolic agent, thermogenic agent, weight loss stimulant, nutrient partitioning modulator, or is a substance which is ingested not directly for its food value, but to interact with the body chemistry to prevent other substances with food value from being eaten, to cause other substances with food value to be eliminated without being absorbed, to cause the body to burn more fat or carbohydrate, or to cause the body to metabolize food differently.

4. Preservatives, Color Enhancers, Thickening Agents, Vitamins, Minerals, etc.

Small amounts of additional ingredients 150 may be added as known in the food art. These are conventional and thus will not be discussed in detail. Typical preservatives include calcium disodium EDTA, sorbic acid, citric acid, potassium sorbate, sodium benzoate, though any preservative may be used that is approved for use in foods. Color enhancers include food coloring and beet juice, though any conventional coloring used in foods may be used. Thickening agents include xanthan gum, guar gum, and karaya gum, though any thickening agent used in foods may be included. Preferred vitamins include vitamin A, vitamin B, vitamin C, vitamin D, vitamin E, and folic acid, but any vitamin may be included. Minerals include phosphorus, magnesium, manganese, copper, zinc, iron, calcium, and potassium, though any mineral may be included. Other ingredients can include flavorings and sweeteners such as sugar, maltodextrin, and sucralose. The composition of the weight loss salad dressing according to the invention may also include inactive excipients, carriers, diluents, adjuvants, and lubricants. Non-limiting examples of inactive excipients, carriers, diluents, lubricants, and adjuvants which can be used in the composition of the present invention include: cellulose, substituted cellulose, calcium carbonate, dicalcium phosphate, starches, lactose, modified food starches, dextrose, calcium sulfate, magnesium carbonate, magnesium stearate, stearic acid, glycerin, polysorbates, lecithin, silicium dioxide, food glaze, talc, croscarmellose sodium, povidone, and gelatin. Additional inactive excipients, carriers, diluents, lubricants and adjuvants which may be used with the active-ingredient composition of this invention are disclosed in the Handbook of Food Additives (CRC Press), which is incorporated by reference herein in relevant part. All of the ingredients in this paragraph can be present in any conventional amount.

As an example of additional ingredients, for the formulation of Table IX, other ingredients may include guar gum, maltodextrin, sucralose, citric acid, and flavorings.

A feature of the invention is that most weight loss supplements, and in particular the preferred weight loss supplements discussed above, taste somewhat acidic, and thus blend well with salad dressings and taste surprisingly good on salads. This good taste is important, as it will encourage the use of the weight loss salad dressing and thus lead to a weight loss program that is easier to follow than prior art programs.

The acidic nature of the weight loss supplements is not so strong as to overwhelm the natural taste of the salad dressing bases given above. However, for very sensitive tastes, in the preferred embodiment, the acidity of salad dressing bases 110 are adjusted when combined with some weight loss supplements 120. For example, the thermogenic supplement in Table V above contains 24 grams of aspartic acid, which is a little more than ¾ ounce, or 1.5 tablespoons of an acid. Thus, if the weight loss supplement of Table V is combined with a salad dressing base, optimally, the acidity of the base should be reduced by about 1.5 tablespoons. For example, if the weight loss supplement of Table V is combined with the Ranch salad dressing base of Table D, preferably, only 1.5 tbls of vinegar should be used instead of the 3 tbls indicated in the table. If it is combined with the Bleu Cheese salad dressing of Table E, which is already highly acidic, an adjustment might not be made; however, for discriminating tastes, the vinegar would be reduced by 1.5 tbls or the lactic acid eliminated altogether. Similarly, an adjustment might or might not be made for the other salad dressing bases. Similarly, the acidity of the salad dressing base may be adjusted for other acidic weight loss supplements, such as that of Table IX. As another example, if the weight loss supplement includes L-carnitine L-tartrate, some people may prefer that an adjustment of the salad dressing base be made. However, it has been found that this particular ingredient blends especially well with salad dressings.

The ingredients are mixed at 160. Any one or more of the salad dressing bases A through G above may be mixed with any one or more of the weight loss supplements I through VIII above to arrive at a weight loss salad dressing. The mixing is preferably done by adding dry ingredients to oil while stirring and then adding other ingredients while continuing to stir, though the order of mixing is not crucial, particularly when using present day mixing apparatus. As is known in the art, the amounts of the various ingredients can be varied.

Since foods, and in particular salad dressings, are quite varied, it is possible that one or more of the weight loss ingredients may have been accidentally included in a salad dressing without the intent of making a weight loss salad dressing. Thus, it is important to the invention that the ingredients described as weight loss supplements be present in the weight loss salad dressings in amounts greater than that present naturally in foods, and more particularly in amounts sufficient to effect weight loss in a human being.

The invention is intended to pertain to all uses of salad dressings. For example, salad dressings are commonly used in sandwich preparation. The fact that the salad dressing may be used on some food other than a salad is contemplated by the invention.

There has been described a novel weight reduction salad dressing, as well as novel systems and methods for weight reduction. Now that the weight loss salad dressing has been described, those skilled in the culinary and weight loss arts may make many variations. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiments described, without departing from the inventive concepts. It is also evident that the methods recited may, in many instances, be performed in a different order; or equivalent ingredients and processes may be substituted for the various ingredients and processes described. Consequently, the invention is to be construed as embracing each and every novel feature and novel combination of features present in and/or possessed by the invention herein described.

1. A weight loss salad dressing comprising:
a salad dressing base; and
a weight loss supplement, which weight loss supplement is present in an amount in excess of the amount of said supplement present naturally in foods.

2. A weight loss salad dressing as in claim 1 wherein said weight loss supplement comprises a fat blocker.

3. A weight loss salad dressing as in claim 1 wherein said weight loss supplement comprises a carbohydrate blocker.

4. A weight loss salad dressing as in claim 1 wherein said weight loss supplement comprises a fat and carbohydrate blocker.

5. A weight loss salad dressing as in claim 1 wherein said weight loss supplement comprises an appetite suppressant.

6. A weight loss salad dressing as in claim 1 wherein said weight loss supplement comprises a metabolizer.

7. A weight loss salad dressing as in claim 1 wherein said weight loss supplement comprises a thermogenic agent.

8. A weight loss salad dressing as in claim 1 wherein said weight loss supplement comprises a weight loss stimulant.

9. A weight loss salad dressing as in claim 1 wherein said weight loss supplement comprises a nutrient partitioning modulator.

10. A weight loss salad dressing as in claim 1 wherein said weight loss supplement comprises mahuang or a derivative thereof.

11. A weight loss salad dressing as in claim 1 wherein said weight loss supplement comprises guarana seed or a derivative thereof.

12. A weight loss salad dressing as in claim 1 wherein said salad dressing base comprises a salad dressing base selected from the group consisting of: an oil and vinegar salad dressing base, a Caesar salad dressing base, a French salad dressing base, a Ranch salad dressing base, a Bleu Cheese salad dressing base, a Russian salad dressing base, and a Thousand Island salad dressing base.

13. A weight loss salad dressing as in claim 1 and further including one or more ingredients selected from a preservative, a color enhancer, a thickening agent, a vitamin, a mineral, and an inactive ingredient.

14. A weight loss salad dressing as in claim 1 wherein the acidity of said salad dressing base is adjusted for any acidity of said weight loss supplement.

15. A salad dressing as in claim 1 wherein said weight loss supplement comprises:
an effective amount of hydroxycitric acid;
an effective amount of carnitine;
an effective amount of biotin;
an effective amount of one or more gluconeogenic substrates selected from the group consisting of: aspartate, lactate, glycerol, and a gluconeogenic amino acid or an alphaketo analogue thereof.

16. A salad dressing as in claim 15 wherein the said weight loss supplement comprises: a weight ratio of the hydroxycitric acid to the carnitine of from about 1:10 to about 100:1; a weight ratio of the hydroxycitric acid to the gluconeogenic substrate of from about 5:1 to about 1:60; and a weight ratio of the hydroxycitric acid to the biotin of from about 50:1 to about 2500:1.

17. A salad dressing as in claim 15 wherein the gluconeogenic amino acid is selected from the group consisting of: alanine, arginine, asparagine, cystine, glutamine, glycine, histidine, hydroxyproline, methionine, proline, serine, threonine, and valine.

18. A salad dressing as in claim 15 wherein said weight loss supplement further comprises an effective amount of at least one nutritional supplement.

19. A salad dressing as in claim 18 wherein the nutritional supplement is selected from the group consisting of: chromium, conjugated linoleic acid, coenzyme Q10, eicosapentaenoic acid, pyridoxine, alpha-lipoic acid, magnesium, and gymnema sylvestre.

20. A salad dressing as in claim 19 wherein said weight loss supplement comprises: from about 0.2 grams to about 8 grams of hydroxycitric acid; from about 10 milligrams to about 10 grams of carnitine; from about 1 gram to about 75 grams of the gluconeogenic substrate; from about 1 milligram to about 25 milligrams of biotin; from about 100 micrograms to about 2 milligrams of chromium; from about 5 milligrams to about 500 milligrams of coenzyme Q10; from about 50 milligrams to about 20 grams of conjugated linoleic acid; from about 10 milligrams to about 10 grams of eicosapentaenoic acid; from about 25 milligrams to about 400 milligrams of pyridoxine; from about 25 milligrams to about 2000 milligrams of alpha lipoic acid; from about 200 milligrams to about 1600 milligrams of magnesium; and from about 20 milligrams to about 2000 milligrams of gymnemic acid.

21. A salad dressing as in claim 19 wherein said weight loss supplement comprises: from about 0.5 grams to about 5 grams of hydroxycitric acid; from about 50 milligrams to about 5 grams of carnitine; from about 1 gram to about 30 grams of the gluconeogenic substrate; from about 2 milligrams to about 10 milligrams of biotin; from about 400 micrograms to about 2400 micrograms of chromium; from about 20 milligrams to about 300 milligrams of coenzyme Q10; from about 1 gram to about 10 grams of conjugated linoleic acid; from about 50 milligrams to about 5000 milligrams of eicosapentaenoic acid; from about 50 milligrams to about 300 milligrams of pyridoxine; from about 50 milligrams to about 1200 milligrams of alpha lipoic acid; from about 400 milligrams to about 1200 milligrams of magnesium; and from about 75 milligrams to about 500 milligrams of gymnemic acid.

22. A weight loss salad dressing as in claim 1 wherein said weight loss supplement comprises L-aspartic acid.

23. A weight loss salad dressing as in claim 1 wherein said weight loss supplement comprises L-carnitine.

24. A weight loss salad dressing as in claim 1 wherein said weight loss supplement comprises garcinia cambogia or a derivative thereof.

25. A weight loss salad dressing as in claim 1 wherein said weight loss supplement comprises garcinia camobogia or a derivative thereof, aspartic acid, L-carnitine, biotin, and chromium.

26. A method of effecting weight loss in a human being, said method comprising ingesting, for a therapeutically effective period of time, an effective amount of a weight loss salad dressing comprising a salad dressing base and a weight loss supplement, which weight loss supplement is present in an amount in excess of the amount of said supplement present naturally in foods.

27. A method as in claim 26, further including said human following a dietary regimen involving a glycemic index of less than 60 and daily calorie consumption comprising less than 50% of calories from carbohydrate intake and at least 20% of calories from protein intake.

28. A method as in claim 26 wherein the method further includes said human following an exercise program involving aerobic and resistance training.

29. A method as in claim 26 wherein the method further involves said human donating blood so as to produce a fall in serum ferritin levels and iron stores.

30. A method as in claim 26 wherein the method further involves said human following a stress reduction program so as to diminish glycocorticoid activity.
